(19) United States
(12) Patent Application Publication
(10) Pub. No.: US 2024/0200126 A1
(43) Pub. Date: Jun. 20, 2024

(54) PRIMER GROUP AND METHOD FOR DETECTING SINGLE-BASE MUTATIONS

(71) Applicant: NANJING GENSCRIPT BIOTECH CO., LTD., Jiangsu (CN)

(72) Inventors: Feichi HU, Jiangsu (CN); Qi WANG, Jiangsu (CN); Cheng-Hsien WU, Jiangsu (CN); Nileshi SARAF, Jiangsu (CN)

(21) Appl. No.: 18/287,245

(22) PCT Filed: Apr. 20, 2022
(86) PCT No.: PCT/CN2022/087791

§ 371 Date: Oct. 17, 2023

(30) Foreign Application Priority Data
Apr. 20, 2021 (CN) 202110425621.4

(51) Int. Cl. C12Q 1/6858; C12Q 1/6876

(52) U.S. Cl. CPC C12Q 1/6858; C12Q 1/6876

(57) ABSTRACT

A primer group and method for detecting single-base mutations. The primer group comprises the following primers: an identification primer, which is composed of, from the 5′ end to the 3′ end, (a) a nucleotide sequence which complements a segment of continuous nucleotides in a nucleic acid sequence to be detected, wherein the 5′ end of the continuous nucleotides starts at a first nucleotide downstream of an expected mutation site; and (b) a nucleotide which complements a non-mutated nucleotide or an expected mutated nucleotide at the expected single-base mutation site of the nucleic acid sequence. The primer group also comprises an amplification primer. The amplification primer is capable of using the identification primer to amplify an amplification product which is obtained by amplifying the nucleic acid sequence. The identification primer is 1 to 19 nucleotides less than the amplification primer.

### CROSS REFERENCE TO RELATED APPLICATION

The present application claims the right of priority for Chinese Patent Application No. 202110425621.4 filed on Apr. 20, 2021, the entire contents of which are incorporated herein by reference.

### TECHNICAL FIELD

The present application relates to the field of biological detection, in particular, to a primer group and method for detecting single-base mutations.

### BACKGROUND ART

The single-base mutation detection of a nucleic acid is very important. It can not only evaluate nucleic acid quality, but more importantly, can be used for the single nucleotide typing detection. Currently, a very large number of diseases are closely related to single-base mutations. If these mutated genes can be effectively identified and detected, early warning of the diseases can be achieved, which facilitates early treatment thereof. Accurate identification of these genes relies on single-base mutation detection techniques. Existing methods for detecting single-base mutations include a sequencing method, a microarray method, a mass spectrometry method, a melting curve method, a Taqman method, etc. The sequencing method, as a gold standard for single nucleotide polymorphism (SNP) analysis, can be used to detect known SNPs and discover unknown SNPs. However, in the sequencing method, each site of each sample needs to be subjected to amplification by a polymerase chain reaction (PCR), gel running, purification by gel slices and then sequencing, which involve many and scattered steps, high cost and an enormous amount of workload, so the sequencing method is both time-consuming and expensive and thus unsuitable for the detection involving a large number of samples and multiple sites. The microarray method has high throughput and is suitable for genome-wide SNP scanning, but its accuracy is relatively low, and a second method needs to be used for verification. The mass spectrometry method is fast and convenient and requires very little sample volume, but it involves a complicated pretreatment process and is suitable for the detection of specific SNPs that have been optimized, not for the detection of new SNPs that have not been optimized. The melting curve method has high throughput and is simple and convenient; however, there are few instruments available for the melting curve method, and the method has high technical requirements and requires professional operation.

Because the above methods involve multi-step reactions, high time costs and high technical requirements, the one-step rapid detection method is highly favored. The Taqman method is a one-step reaction method, which mainly relies on the selectivity of specific enzymes and high-cost fluorescent molecular modifications for the single-base mutation detection. In addition, the existing Taqman method improves the selectivity of the method mainly by means of artificially introducing mismatched bases in a primer sequence design and performing enzyme improvement technologies; however, the introduction of improperly mismatched bases may result in incorrect results, which leads to the need for verification by multiple experiments, and the improvement of enzymes is complex and expensive.

Therefore, developing new technologies or designs that can realize simple, fast and low-cost one-step detections is a new direction with market competition values.

### SUMMARY OF THE INVENTION

The present application provides a new method for detecting a single-base mutation of a nucleic acid, wherein the method utilizes two PCR primers with different lengths (a short-chain primer and a long-chain primer), which have different binding capacity to a target sequence to be detected. The short-chain primer can identify and hybridize with a matched target nucleic acid sequence first, which can realize an unbalanced PCR.

With regard to an exemplary schematic diagram of the principle of the method of the present patent, reference can be made to FIG. 1. Specifically, when the nucleotide at the 3′ end of the short-chain primer cannot be correctly paired with the single-base mutation site of the target nucleic acid sequence, the binding capacity of the short-chain primer to the target nucleic acid sequence is greatly weakened, making hybridization difficult, and resulting in no amplification products; and when the nucleotide at the 3′ end of the short-chain primer is correctly paired with the single-base mutation site of the target nucleic acid sequence, an amplification product can be obtained by amplification at a suitable annealing temperature. The long-chain primer has a strong ability to hybridize with a product obtained by amplification using the short-chain primer, and in this case, the product obtained by amplification using the short-chain primer can be normally bound and replicated. After multiple PCR cycles for amplification, the correct specific amplification products generated by the long-chain primer and the short-chain primer are amplified exponentially. After the PCR amplification product is subject to gel electrophoresis, it can be clearly found that when there is a base mutation in a target sequence, no gel electrophoresis band can be observed; and when there is no base mutation, a clear gel electrophoresis band can be observed, thereby realizing a single-base mutation detection of a nucleic acid. If quantitative PCR or DNA chip technology is used, qualitative and quantitative detection of single-base mutations can be achieved. Compared with the existing methods, the method of the present application is easy to operate, low in cost, and does not require harsh reaction conditions.

In a first aspect, the present application provides a method for detecting a single-base mutation in a target nucleic acid sequence. The expression “detecting a single-base mutation in a target nucleic acid sequence” includes detecting whether there is a mutation at an expected single-base mutation site of the nucleic acid sequence and detecting (i.e., identifying) a nucleotide at an expected single-base mutation site of the nucleic acid sequence.

Therefore, the present application provides a method for detecting whether there is a mutation at an expected single-base mutation site of a nucleic acid sequence, the method comprising:
providing a sample containing a nucleic acid sequence to be detected;
amplifying the nucleic acid sequence to be detected in the sample by a polymerase chain reaction using a primer group, wherein the primer group comprises the following primers:
an identification primer comprising from the 5′ end to the 3′ end: (a) a nucleotide sequence which complements a segment of continuous nucleotides in the nucleic acid sequence to be detected, wherein the 5′ end of the continuous nucleotides starts at a first nucleotide downstream of an expected mutation site, and (b) a nucleotide which complements a non-mutated nucleotide at the expected single-base mutation site of the nucleic acid sequence to be detected,
an amplification primer capable of amplifying an amplification product obtained by amplifying the nucleic acid sequence to be detected using the identification primer,
wherein the identification primer is 1 to 19 nucleotides less than the amplification primer; and
detecting whether there is a specific amplification product in a reaction product, wherein the presence of the specific amplification product indicates that there is no single-base mutation at the expected mutation site of the nucleic acid sequence to be detected.

The present application further provides a method for detecting a nucleotide at an expected single-base mutation site of a nucleic acid sequence, the method comprising:
providing a sample containing a nucleic acid sequence to be detected;
amplifying the nucleic acid sequence in the sample by a polymerase chain reaction using a primer group, wherein the primer group comprises the following primers:
an identification primer, which is composed of, from the 5′ end to the 3′ end, (a) a nucleotide sequence which complements a segment of continuous nucleotides in the nucleic acid sequence to be detected, wherein the 5′ end of the continuous nucleotides starts at a first nucleotide downstream of an expected mutation site, and (b) a nucleotide which complements a nucleotide expected to exist at the expected single-base mutation site of the nucleic acid sequence to be detected, and
an amplification primer capable of amplifying an amplification product obtained by amplifying the nucleic acid sequence to be detected using the identification primer,
wherein the identification primer is 1 to 19 nucleotides less than the amplification primer; and
detecting whether there is a specific amplification product in a reaction product, wherein the presence of the specific amplification product indicates that there is the nucleotide expected to exist at the expected mutation site of the nucleic acid sequence to be detected.

In the context of the present application, the “nucleic acid sequence” may be a double-stranded or single-stranded nucleic acid, such as a double-stranded DNA, a single-stranded DNA or RNA.

In the context of the present application, the “single-base mutation” refers to a mutation resulting from the substitution of a single base on a nucleic acid sequence.

In the context of the present application, the term “identification primer” can be used interchangeably with “short-chain primer”, “primer 1” and “short-chain primer 1”. The “identification primer” is a short-chain primer (compared with the length of a conventional PCR primer), which can realize SNP recognition only by the base at the 3′ end, thus avoiding the introduction of a second artificial mismatched base, and ensuring the specificity of the method. In the method for detecting whether there is a mutation at an expected single-base mutation site of a nucleic acid sequence, the “identification primer” is a nucleotide sequence which complements a segment of contiguous nucleotides of a non-mutated nucleic acid sequence, wherein the nucleotide at the 3′ end of the primer correspondingly complements the non-mutated nucleotide at the expected single-base mutation site of the nucleic acid sequence to be detected. In the method for detecting a nucleotide at an expected single-base mutation site of a nucleic acid sequence, the “identification primer” is a nucleotide sequence which complements a segment of continuous nucleotides of an expected mutated nucleic acid sequence, wherein the nucleotide at the 3′ end of the primer correspondingly complements an expected mutated nucleotide at the expected single-base mutation site of the nucleic acid sequence to be detected. For example, if the nucleotide at the expected single-base mutation site of the non-mutated nucleic acid sequence is A, the nucleotide at the 3′ end of the identification primer is T, which is complementary to A, in the method for detecting whether there is a mutation at an expected single-base mutation site of a nucleic acid sequence; and in the method for detecting a nucleotide at an expected single-base mutation site of a nucleic acid sequence, the nucleotide at the 3′ end of the identification primer is a nucleotide which complements an expected mutated nucleotide (e.g., if the expected mutated nucleotide is G, the nucleotide at the 3′ end of the identification primer is C).

In the context of the present application, the term “amplification primer” can be used interchangeably with “long-chain primer”, “primer 2”, and “long-chain primer 2”. The amplification primer is the primer used in a conventional ordinary PCR. An ordinary PCR primer is generally between 15 and 30 nucleotides in length, and a commonly used primer is 18 to 27 nucleotides in length. In the method for detecting a single-base mutation in a target nucleic acid sequence of the present application, the “amplification primer” can complement a segment of continuous nucleotides in the amplification product obtained by amplifying the nucleic acid sequence to be detected using the identification primer. In terms of sequences, the “amplification primer” may be composed of the same continuous nucleotides as a segment of continuous nucleotides in the nucleic acid sequence to be detected.

In the context of the present application, the “expected single-base mutation site” refers to a site on a nucleic acid sequence to be detected with a mutation present or absent, which may be a site known by the prior art to be prone to having a single-base mutation, or may be any nucleotide site to be determined whether there is a single-base mutation.

In the method for detecting a nucleotide at an expected single-base mutation site of a nucleic acid sequence, the “nucleotide expected to exist” at the expected single-base mutation site refers to a nucleotide that may exist at a site to be detected, which may be a nucleotide known by the prior art to be prone to existing at the site, or may be any nucleotide that may exist. For example, in some embodiments, the nucleotides in (b) of the identification primer may be selected from 1, 2, 3 or 4 of A, C, T, and G, and the nucleotides at the sites to be detected can be determined by performing 1, 2, 3 or 4 PCRs simultaneously or sequentially with these primers correspondingly.

In some embodiments of the method for detecting a single-base mutation in a target nucleic acid sequence, the identification primer is 1 to 19 nucleotides less than the amplification primer. In some preferred embodiments, the identification primer is 2 to 16 nucleotides less than the amplification primer, e.g., 2, 3, 4, 5, 6, 7, 8, 9, 10, 11, 12, 13, 14, 15 or 16 nucleotides less than the amplification primer. In other preferred embodiments, the identification primer is 3 to 15 nucleotides less than the amplification primer, e.g., 3, 4, 5, 6, 7, 8, 9, 10, 11, 12, 13, 14 or 15 nucleotides less than the amplification primer. In a most preferred embodiment, the identification primer is 2 to 8 nucleotides less than the amplification primer, e.g., 2, 3, 4, 5, 6, 7 or 8 nucleotides less than the amplification primer.

In some embodiments of the method for detecting a single-base mutation in a target nucleic acid sequence, the identification primer is 11 to 16 nucleotides in length, e.g., 11, 12, 13, 14, 15 or 16 nucleotides in length. In some preferred embodiments, the identification primer is 12 to 15 nucleotides in length. In a specific embodiment, the identification primer is 12 nucleotides in length.

In some embodiments of the method for detecting a single-base mutation in a target nucleic acid sequence, the amplification primer is 15 to 30 nucleotides in length, such as 15 to 27 nucleotides in length, such as 15 to 25 nucleotides in length, such as 15, 16, 17, 18, 19, 20, 21, 22, 23, 24, 25, 26, 27, 28, 29 or 30 nucleotides in length. In a preferred embodiment, the amplification primer is 16 to 20 nucleotides in length. In a specific embodiment, the amplification primer is 20 nucleotides in length.

In some preferred embodiments of the method for detecting a single-base mutation in a target nucleic acid sequence, the identification primer is 12 nucleotides in length and the amplification primer is 20 nucleotides in length, or the identification primer is 15 nucleotides in length and the amplification primer is 20 nucleotides in length, or the identification primer is 13 nucleotides in length and the amplification primer is 20 nucleotides in length, or the identification primer is 14 nucleotides in length and the amplification primer is 20 nucleotides in length.

The amplification reaction (i.e., the polymerase chain reaction) of the method of the present application is performed in an amplification reaction mixture. The mixture contains reagents required to complete a primer extension reaction or nucleic acid amplification, and non-limiting examples of such reagents include primers, polymerases, buffers, cofactors (e.g., divalent or monovalent cations) and nucleotides (e.g., dNTPs).

In the method of the present application, the polymerase chain reaction is performed using a DNA polymerase. The DNA polymerase may be a commonly used DNA polymerase known in the art. In some embodiments, the DNA polymerase is a high-fidelity polymerase. In some embodiments, the DNA polymerase is selected from: a hot-start Taq polymerase, a TaqNova Stoffel DNA polymerase, an HiFi-KAPA polymerase, and an Hemo KlenTaq polymerase, e.g., a DNA polymerase (Hot-start Taq polymerase (E00049, GENSCRIPT BIOTECH CO., LTD.), TaqNova Stoffel DNA polymerase (RP810, BLIRT), HiFi-KAPA polymerase 2× (KK2601, Roche), and Hemo KlenTaq polymerase (M0332S, NEB)). In a preferred embodiment, the DNA polymerase is an HiFi-KAPA polymerase.

In some embodiments of the method of the present application, the polymerase chain reaction may include a pre-denaturation step, a cycle amplification step, and a final extension step, and each cycle in the cycle amplification step may include denaturation, annealing and extension steps. In some embodiments, the cycle amplification step is performed for 18-30 cycles, e.g., 20 cycles. In some embodiments, each cycle in the cycle amplification step is performed at 98° C. for 10 s, at 45-52° C. for 15-30 s, or at 72° C. for 15 s. In some embodiments, the annealing temperature is 44° C. to 52° C., such as 44° C., 45° C., 46° C., 47° C., 48° C., 49° C., 50° C., 51° C. or 52° C., preferably 45° C. to 50° C. In a specific embodiment, the annealing temperature is 45° C. In another specific embodiment, the annealing temperature is 50° C.

In the method of the present application, a step of purifying the amplification product may be involved or not involved after the amplification step and before the detection step. The purification can be performed using nucleic acid purification methods commonly known in the art, such as gel electrophoresis.

In the context of the present application, the “specific amplification product” refers to a product having a specific length amplified by the primer group (i.e., the identification primer and the amplification primer) of the present application. In some embodiments, the specific amplification product is at least 40 nucleotides in length, and may be more than 50 nucleotides in length, such as 70 to 700 nucleotides in length, such as 70 to 120 nucleotides in length.

The detection of the specific amplification product can be performed by a detection method selected from: gel electrophoresis, mass spectrometry, SYBR I fluorescence method, SYBR II fluorescence method, SYBR gold, Pico green, TOTO-3, intercalating dye detection, fluorescence resonance energy transfer (FRET), molecular beacon detection, etc.

In some embodiments of the method of the present application, the polymerase chain reaction is an ordinary PCR, and the reaction product is detected by gel electrophoresis.

In some embodiments of the method of the present application, the polymerase chain reaction is a fluorescent quantitative PCR. For example, the polymerase chain reaction is performed using a fluorescent dye for a fluorescent quantitative PCR, such as SYBR I, SYBR II, or SYBR gold.

In a second aspect, the present application provides a primer group for detecting a single-base mutation in a nucleic acid sequence, the primer group comprising the following primers:
an identification primer, which is composed of, from the 5′ end to the 3′ end, (a) a nucleotide sequence which complements a segment of continuous nucleotides in a nucleic acid sequence to be detected, wherein the 5′ end of the continuous nucleotides starts at a first nucleotide downstream of an expected mutation site; and (b) a nucleotide which complements a non-mutated nucleotide or an expected mutated nucleotide at an expected single-base mutation site of a nucleic acid sequence to be detected;
an amplification primer capable of amplifying an amplification product obtained by amplifying the nucleic acid sequence to be detected using the identification primer, wherein the identification primer is 1 to 19 nucleotides less than the amplification primer.

For some embodiments of the primer group, the identification primer is 2 to 16 nucleotides less than the amplification primer, preferably the identification primer is 3 to 15 nucleotides less than the amplification primer. In other preferred embodiments, the identification primer is 2 to 8 nucleotides less than the amplification primer, e.g., 2, 3, 4, 5, 6, 7 or 8 nucleotides less than the amplification primer.

In a third aspect, the present application provides the use of the primer group of the present application in the preparation of a mixture, a kit or a biological detection device for detecting a single-base mutation in a nucleic acid sequence.

In a fourth aspect, the present application provides a mixture comprising the primer group of the present application, a DNA polymerase, and a nucleic acid sequence to be detected. In some embodiments, the mixture further comprises a reagent for detecting an amplification product, such as SYBR I, SYBR II, or SYBR Gold. In some embodiments, the mixture further comprises other reagents required to complete a primer extension reaction or nucleic acid amplification, such as buffers, cofactors (e.g., divalent or monovalent cations) and nucleotides (e.g., dNTPs).

In a fifth aspect, the present application provides a kit for detecting a single-base mutation in a nucleic acid sequence, comprising the primer group of the present application. In some embodiments, the kit further comprises a DNA polymerase. In some embodiments, the kit further comprises a reagent for detecting an amplification product, such as SYBR I, SYBR II, or SYBR Gold. In some embodiments, the kit further comprises reagents and/or materials required for nucleic acid immobilization, hybridization, and/or detection, such as solid supports (e.g., multi-well plates), buffers and nucleic acid standards. In some embodiments, the kit comprises a nucleic acid chip. In some embodiments, the kit further comprises instructions for use in the method of the present application.

In a sixth aspect, the present application provides a biological detection device for detecting a single-base mutation in a nucleic acid sequence, comprising the primer group of the present application. Non-limiting examples of the detection devices include a microfluidic device.

The features, definitions and preferences described in the first aspect apply equally to the second to sixth aspects.

### BRIEF DESCRIPTION OF THE DRAWINGS

The present invention is described in more detail with reference to the following figures.

FIG. 1 exemplarily shows the principle of the unbalanced PCR method of the present application, wherein \textcircled{1} represents the nucleic acid sequence to be detected, \textcircled{2} represents the identification primer, and \textcircled{3} represents the amplification primer.

FIG. 2 shows a gel electrophoretogram of PCR products from target sequences using combinations of different lengths of short-chain primers and long-chain primers, wherein lane a shows the PCR product of 11 nt+20 nt primers, lane b shows the PCR product of 12 nt+20 nt primers, lane c shows the PCR product of 11 nt+12 nt primers, lane d shows the PCR product of 12 nt+12 nt primers, and the far right shows the molecular weight marker (the molecular weight from top to bottom is 3000, 2000, 1500, 1000, 700, 500, 250, and 100 bp).

FIG. 3 shows a gel electrophoretogram of PCR products from target sequences using a combination of a short-chain primer (15 nt) and a long-chain primer (20 nt), wherein lane 1 shows the target sequence, lane 2 shows the target sequence having a single-base mutation, and the far left shows the molecular weight marker (the molecular weight of bands from top to bottom is 1031, 900, 800, 700, 600, 500, 400, 300, 250, 200, 150, 100, and 50 bp).

FIG. 4 shows a gel electrophoretogram of PCR products obtained at different annealing temperatures, wherein lane 1 shows the target sequence, lane 2 shows the target sequence having a single-base mutation, and lane 3 shows the molecular weight marker (the molecular weight from top to bottom is: 1031, 900, 800, 700, 600, 500, 400, 300, 250, 200, 150, 100, and 50 bp in the panel for 45° C.; and 3000, 2000, 1500, 1000, 700, 500, 250, and 100 bp in the panels for 50° C., 51° C. and 52° C.).

FIG. 5 shows a comparative experiment of detecting single-base mutations by an unbalanced PCR and a conventional PCR, wherein lane 1 shows the product obtained by detecting the target sequence by the conventional PCR method, lane 2 shows the product obtained by detecting the single-base mutation sequence by the conventional PCR method, lane 3 shows the product obtained by detecting the target sequence by the unbalanced PCR, lane 4 shows the product obtained by detecting the single-base mutation sequence by the unbalanced PCR, and the last lane shows the molecular weight marker (the molecular weight from top to bottom is 3000, 2000, 1500, 1000, 700, 500, 250, and 100 bp).

FIG. 6 shows repeatability experiments of the method of the present invention, wherein lane 1 shows the PCR result with the target sequence as a template, lane 2 shows the PCR result with the single-base mutation sequence as a template, and lane 3 shows the molecular weight marker (the molecular weight from top to bottom is 3000, 2000, 1500, 1000, 700, 500, 250, and 100 bp).

FIG. 7 shows a gel electrophoretogram of purified PCR products and concentrations thereof, wherein lanes 1 and 3 show purified PCR products with the target sequence as a template, lanes 2 and 4 show purified PCR products with the single-base mutation sequence as a template, and the last lane shows the molecular weight marker (the molecular weight from top to bottom is 3000, 2000, 1500, 1000, 700, 500, 250, and 100 bp).

FIG. 8 shows the detection of unknown mutants of single-base mutations using the unbalanced PCR, wherein lane 1 shows the result using SEQ ID NO: 3 as primer 1, lane 2 shows the result using SEQ ID NO: 4 as primer 1, lane 3 shows the result using SEQ ID NO: 5 as primer 1, lane 4 shows the result using SEQ ID NO: 6 as primer 1, lane 5 shows a blank control in which the target sequence is replaced with water, and the leftmost lane shows the molecular weight marker (the molecular weight from top to bottom is 3000, 2000, 1500, 1000, 700, 500, 250, and 100 bp).

FIG. 9 shows the detection of single-base mutations by a real-time fluorescent quantitative PCR, wherein panel a shows an amplification curve graph, and panel b shows a melting curve graph.

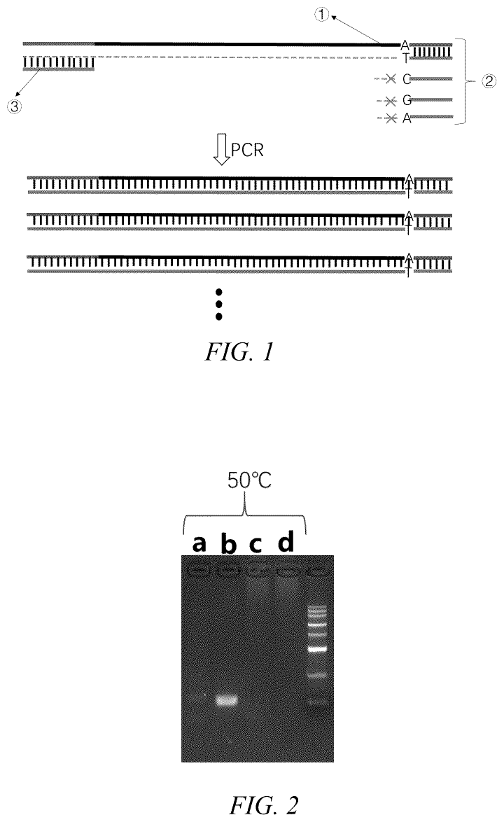

### DETAILED DESCRIPTION OF EMBODIMENTS

Unless defined otherwise, the technical and scientific terms used herein have the same meaning as commonly understood by a person skilled in the art to which the present invention belongs.

The technical solutions of the present invention are further illustrated in more detail by the examples and in conjunction with the accompanying drawings. Unless otherwise specified, the methods and materials in the examples described below are conventional products that can be purchased from the market. Those skilled in the art of the present invention would understand that the methods and materials described below are exemplary only and should not be considered as limiting the scope of the present invention.

### Example 1: Detection of Single-Base Mutations Using Unbalanced PCR

### 1.1 Primer Design

The following sequences and primers were all synthesized by NANJING GENSCRIPT BIOTECH CO., LTD.

The target sequence to be detected is 5′-CTTTACTTACTACACCTCAGATATATTTCTTCATGAAGACCTCACAGTAAAAAT AGGTGATTTTGGTCTAGCTACAGAAGAAATCTCGATGGAGTGGG (SEQ ID NO: 1). The sequence having a single-base mutation relative to the target sequence is 5′-CTTTACTTACTACACCTCAGATATATTTCTTCATGAAGACCTCACAGTAAAAAT AGGTGATTTTGGTCTAGCTACAGATGAAATCTCGATGGAGTGGG (SEQ ID NO: 2).

Short-chain primer 1 is a specific primer for detecting whether there is a single-base mutation, and the last base at the 3′ end of the sequence hybridizes to the mutation site in the target sequence. A total of 5 sets of short-chain primers 1 with bases of 11 nt, 12 nt, 13 nt, 14 nt and 15 nt were designed, and the sequences are shown in Table 1 below.

TABLE 1
5 sets of short-chain primers 1 with bases of
11 nt, 12 nt, 13 nt, 14 nt and 15 nt

| Primer | |
|---|---|
| name | Sequence |
| 1-11 nt-1 | 5′-TCGAGATTTCT (SEQ ID NO: 23) |
| 1-11 nt-2 | 5′-TCGAGATTTCA (SEQ ID NO: 24) |
| 1-11 nt-3 | 5′-TCGAGATTTCG (SEQ ID NO: 25) |
| 1-11 nt-4 | 5′-TCGAGATTTCC (SEQ ID NO: 26) |
| 1-12 nt-1 | 5′-ATCGAGATTTCT (SEQ ID NO: 3) |
| 1-12 nt-2 | 5′-ATCGAGATTTCA (SEQ ID NO: 4) |
| 1-12 nt-3 | 5′-ATCGAGATTTCG (SEQ ID NO: 5) |
| 1-12 nt-4 | 5′-ATCGAGATTTCC (SEQ ID NO: 6) |
| 1-13 nt-1 | 5′-CATCGAGATTTCT (SEQ ID NO: 7) |
| 1-13 nt-2 | 5′-CATCGAGATTTCA (SEQ ID NO: 8) |
| 1-13 nt-3 | 5′-CATCGAGATTTCG (SEQ ID NO: 9) |
| 1-13 nt-4 | 5′-CATCGAGATTTCC (SEQ ID NO: 10) |
| 1-14 nt-1 | 5′-CCATCGAGATTTCT (SEQ ID NO: 11) |
| 1-14 nt-2 | 5′-CCATCGAGATTTCA (SEQ ID NO: 12) |
| 1-14 nt-3 | 5′-CCATCGAGATTTCG (SEQ ID NO: 13) |
| 1-14 nt-4 | 5′-CCATCGAGATTTCC (SEQ ID NO: 14) |
| 1-15 nt-1 | 5′-TCCATCGAGATTTCT (SEQ ID NO: 15) |
| 1-15 nt-2 | 5′-TCCATCGAGATTTCA (SEQ ID NO: 16) |
| 1-15 nt-3 | 5′-TCCATCGAGATTTCG (SEQ ID NO: 17) |
| 1-15 nt-4 | 5′-TCCATCGAGATTTCC (SEQ ID NO: 18) |

Long-chain primer 2 is a universal primer that hybridizes to an amplification product of short-chain primer 1. A total of two long-chain primers 2 with bases of 12 nt and 20 nt were designed, and the sequences are shown in Table 2 below.

TABLE 2
Two long-chain primers 2 with bases of 12 nt and
20 nt

| Primer | |
|---|---|
| name | Sequence |
| 2-12 nt | 5′-CTTTACTTACTA (SEQ ID NO: 22) |
| 2-20 nt | 5′-CTTTACTTACTACACCTCAG (SEQ ID NO: 19) |

### 1.2 Unbalanced PCR Amplification

The PCR system was 20 μL, including 7 μL of ddH2O, 1 μL of short-chain primer 1 (10 μmol·L⁻¹) designed in example 1.1, 1 μL of long-chain primer 2 (10 μmol·L⁻¹) designed in example 1.1, 1 μL of template DNA (the target sequence or the single-base mutation sequence) (1 nmol·L⁻¹), and 10 μL of HiFi-KAPA polymerase 2× (KK2601, Roche).

PCRs were performed on a Biometra T1 thermolcycler (C1000 Touch, Bio-Rad). The reaction conditions involved: pre-denaturation at 98° C. for 30 s; denaturation at 98° C. for 10 s, annealing at a particular temperature (45° C., 50° C., 51° C. or 52° C.) for 30 s, and extension at 72° C. for 15 s, 20 cycles; and extension at 72° C. for 5 min.

### 1.3 Gel Electrophoresis Test

2 μL of PCR products from example 1.2 were taken and mixed well with 0.5 μL of a dye for plasmid mass extraction (Goldview, SBS GENETECH CO., LTD.), the resulting mixed solution was added into the well of 2.5% agarose gel (Invitrogen) for gel electrophoresis, and the gel image (DYY-8C type, BEIJING LIUYI BIOTECHNOLOGY CO., LTD; 120V, 20 min) was collected.

### 1.4 Results

When the PCR annealing temperature was selected as 50° C., the results of subjecting the target sequence to PCR amplification by using combinations of different lengths of primers 1 (1 nt and 12 nt) and different lengths of primers 2 (12 nt and 20 nt) were tested. The specific reaction primers and results are shown in Table 3 below, and the electropherogram by subjecting reaction products to agarose gel electrophoresis are shown in FIG. 2. It can be seen that the target sequence band can be well amplified using the 12 nt short-chain primer 1 and the 20 nt long-chain primer 2.

TABLE 3

| | | | Whether the target sequence |
|---|---|---|---|
| | Short-chain primer 1 | Long-chain primer 2 | band appears |
| a | 11 nt (SEQ ID NO: 23) | 20 nt (SEQ ID NO: 19) | Yes (weak but identifiable) |
| b | 12 nt (SEQ ID NO: 3) | 20 nt (SEQ ID NO: 19) | Yes |
| c | 11 nt (SEQ ID NO: 23) | 12 nt (SEQ ID NO: 22) | No |
| d | 12 nt (SEQ ID NO: 3) | 12 nt (SEQ ID NO: 22) | No |

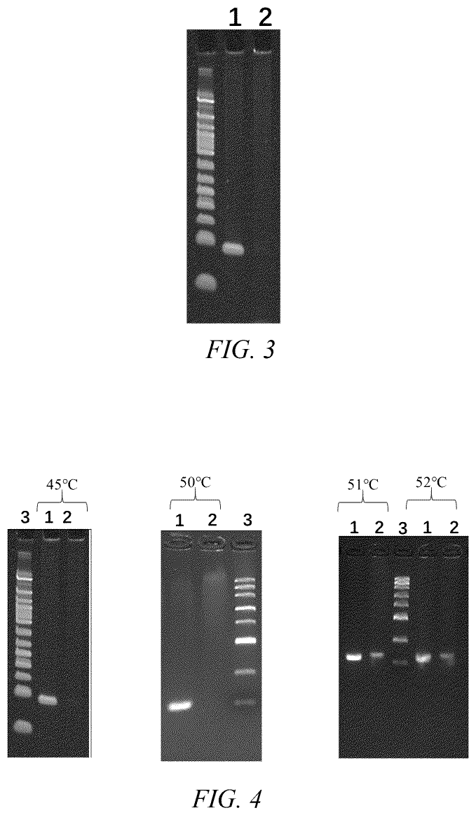

When the PCR annealing temperature was selected as 50° C., the results of subjecting the target sequence (SEQ ID NO: 1) and the sequence (SEQ ID NO: 2) having a single-base mutation relative to the target sequence to PCR amplification by using the 15 nt short-chain primer 1 (SEQ ID NO: 15) and the 20 nt long-chain primer 2 (SEQ ID NO: 19) were tested. FIG. 3 shows an electropherogram by subjecting reaction products to agarose gel electrophoresis, wherein lane 1 shows the result with the target sequence as a template, and lane 2 shows the result with a sequence having a single-base mutation relative to the target sequence as a template. It can be seen that the target sequence band can also be amplified using a combination of short-chain primer 1 (15 nt) and long-chain primer 2 (20 nt), which cannot amplify the sequence having a single-base mutation relative to the target sequence.

PCRs were performed by selecting the 12 nt primer 1 (SEQ ID NO: 3) and the 20 nt primer 2 (SEQ ID NO: 19) and at different annealing temperatures (45° C., 50° C., 51° C., and 52° C.). The electropherogram from agarose gel electrophoresis is shown in FIG. 4, wherein lane 1 shows the result with the target sequence as a template, and lane 2 shows the result with a sequence having a single-base mutation relative to the target sequence as a template. It can be seen that 45° C. and 50° C. as annealing temperatures can achieve better effects, and 51° C. and 52° C. can also be effective as annealing temperatures.

### Example 2: Comparative Experiment of Detecting Single-Base Mutations by Unbalanced PCR and Conventional PCR

### 1.1 Unbalanced PCR

The target sequence to be detected was SEQ ID NO: 1 in example 1, the sequence having a single-base mutation relative to the target sequence was SEQ ID NO: 2 in example 1, the short-chain primer 1 used was 5′-ATCGAGATTTCT (SEQ ID NO: 3), and the long-chain primer 2 used was 5′-CTTTACTTACTACACCTCAG (SEQ ID NO: 19).

PCR amplification conditions were as follows: the reaction system was 20 μL, including 7 μL of ddH₂O, 1 μL of primer 1 (10 μmol·L⁻¹), 1 μL of primer 2 (10 μmol·L⁻¹), 1 μL of template DNA (the target sequence or the sequence having a single-base mutation relative to the target sequence) (1 nmol·L⁻¹), and 10 μL of HiFi-KAPA polymerase 2×.

PCRs were performed on a Biometra T1 thermolcycler. The reaction conditions involved: pre-denaturation at 98° C. for 30 s; denaturation at 98° C. for 10 s, annealing at a particular temperature (45° C. or 50° C.) for 30 s, and extension at 72° C. for 15 s, 20 cycles; and extension at 72° C. for 5 min.

### 1.2 Conventional PCR

The target sequence to be detected was SEQ ID NO: 1 in example 1, the sequence having a single-base mutation relative to the target sequence was SEQ ID NO: 2 in example 1, and the primers used were conventional PCR primer 1 (CCCACTCCATCGAGATTTCT, SEQ ID NO: 20) and conventional PCR primer 2 (CTTTACTTACTACACCTCAG, SEQ ID NO: 21).

PCR amplification conditions were as follows: the reaction system was 20 μL, including 7 μL of ddH₂O, 1 μL of conventional PCR primer 1 (10 μmol·L⁻¹), 1 μL of conventional PCR primer 2 (10 μmol·L⁻¹), 1 μL of template DNA (the target sequence or the sequence having a single-base mutation relative to the target sequence) (1 nmol·L⁻¹), and 10 μL of HiFi-KAPA polymerase 2×.

PCRs were performed on a Biometra T1 thermolcycler (C1000 Touch, Bio-Rad). The reaction conditions involved: pre-denaturation at 98° C. for 30 s; denaturation at 98° C. for 10 s, annealing at 50° C. for 30 s, and extension at 72° C. for 15 s, 20 cycles; and extension at 72° C. for 5 min.

3. Gel electrophoresis test: 2 μL of PCR product samples from examples 2.1 and 2.2 were respectively taken and mixed well with 0.5 μL of a dye for plasmid mass extraction (Goldview, SBS GENETECH CO., LTD.), the resulting mixed solution was added into the well of 2.5% agarose gel (Invitrogen) for gel electrophoresis, and the gel image (DYY-8C type, BEIJING LIUYI BIOTECHNOLOGY CO., LTD; 120V, 20 min) was collected.

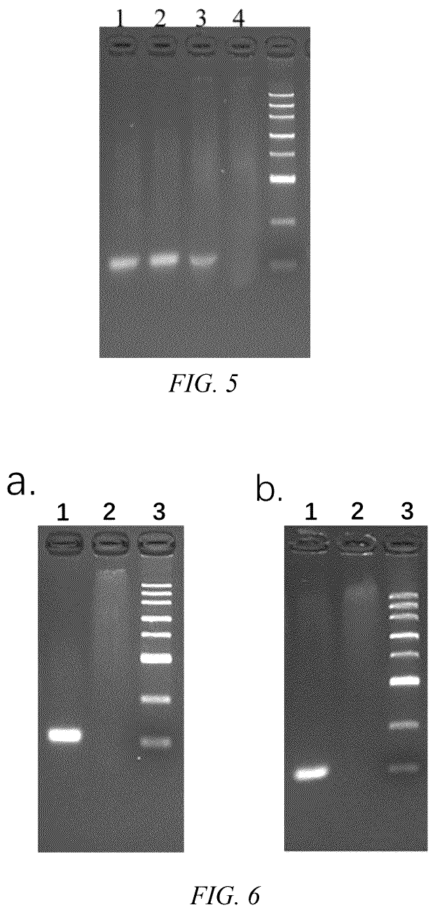

As shown in FIG. 5, lane 1 shows the product obtained by detecting the target sequence by the conventional PCR method, lane 2 shows the product obtained by detecting the single-base mutation sequence by the conventional PCR method, lane 3 shows the product obtained by detecting the target sequence by the unbalanced PCR, lane 4 shows the product obtained by detecting the single-base mutation sequence by the unbalanced PCR, and It can be seen that when using the conventional PCR method, obvious bands were observed for both the single-base mutation sequence and the target sequence as templates, indicating that the use of conventional PCR primers is easy to cause a non-specific amplification. When using the unbalanced PCR method, obvious bands were observed only for the target sequence, and no bands were observed for the single-base mutation sequence, indicating that the unbalanced PCR method can accurately identify single-base mutations and reduce the non-specific amplification.

### Example 3: Repeatability of Detecting Single-Base Mutations by Unbalanced PCR

The target sequence to be detected was SEQ ID NO: 1 in example 1, the sequence having a single-base mutation relative to the target sequence was SEQ ID NO: 2 in example 1, the short-chain primer 1 used was 5′-ATCGAGATTTCT (SEQ ID NO: 3), and the long-chain primer 2 used was 5′-CTTTACTTACTACACCTCAG (SEQ ID NO: 19).

PCR amplification conditions were as follows: the reaction system was 20 μL, including 7 μL of ddH₂O, 1 μL of primer 1 (10 μmol·L⁻¹), 1 μL of primer 2 (10 μmol·L⁻¹), 1 μL of template DNA (the target sequence or the sequence having a single-base mutation relative to the target sequence) (1 nmol·L⁻¹), and 10 μL of HiFi-KAPA polymerase 2×.

PCRs were performed on a Biometra T1 thermolcycler. The reaction conditions involved: pre-denaturation at 98° C. for 30 s; denaturation at 98° C. for 10 s, annealing at 50° C. for 30 s, and extension at 72° C. for 15 s, 20 cycles; and extension at 72° C. for 5 min.

Gel electrophoresis test: 2 μL of PCR product samples were mixed well with 0.5 μL of a dye for plasmid mass extraction (Goldview, SBS GENETECH CO., LTD.), the resulting mixed solution was added into the well of 2.5% agarose gel (Invitrogen) for gel electrophoresis, and the gel image (DYY-8C type, BEIJING LIUYI BIOTECHNOLOGY CO., LTD; 120V, 20 min) was collected.

As shown in FIG. 6a, lane 1 shows the PCR result with the target sequence as a template, and a bright band can be observed; lane 2 shows the PCR result with the single-base mutation sequence as a template, and no obvious band can be observed; and lane 3 shows the molecular weight marker. It can be seen that the single-base mutations can be clearly identified by the method of the present invention. FIG. 6b shows a repeatability experiment performed under the same conditions as FIG. 6a, and it can be seen from the obtained gel electrophoretogram that the method of the present invention has good repeatability.

### Example 4: Purification and Concentration Determination of Unbalanced PCR Product

The PCR products obtained in the unbalanced PCR of example 2 were purified by smart beads (YEASEN BIOTECHNOLOGY (SHANGHAI) CO., LTD.), and the following operations were performed according to the instructions provided by the manufacturer: 1) magnetic beads were taken out of the refrigerator and equilibrated at room temperature for at least 30 minutes; 2) the magnetic beads were vortexed or inverted thoroughly to ensure thorough mixing; 3) 1. Ox Hieff NGS® Smarter DNA Clean Beads were transferred into a DNA solution (EP tube for PCR product) and incubated at room temperature for 5 minutes; 4) the PCR tube was briefly centrifuged and placed in a magnetic rack to separate the magnetic beads and liquid; after the solution became clear (about 5 minutes), the supernatant was carefully removed; 5) with the PCR tube kept in the magnetic rack, the magnetic beads were rinsed by adding 200 μL of freshly prepared 80% ethanol, and incubated at room temperature for 30 seconds before the supernatant was carefully removed; 6) step 5 was repeated for a total of 2 rinses; 7) with the PCR tube kept in the magnetic rack, the cap was opened for air drying the magnetic beads until cracks appeared (about 5 minutes); 8) the PCR tube was taken out of the magnetic rack, 21 μL of ddH₂O was added, and the resultant was gently pipetted with a pipette until fully mixed, and left to stand at room temperature for 5 minutes; and 9) the PCR tube was briefly centrifuged, placed in the magnetic rack and left to stand; after the solution became clear (about 5 minutes), 20 μL of the supernatant was carefully pipetted into a new PCR tube without touching the magnetic beads, and a pure double-strand DNA product was obtained.

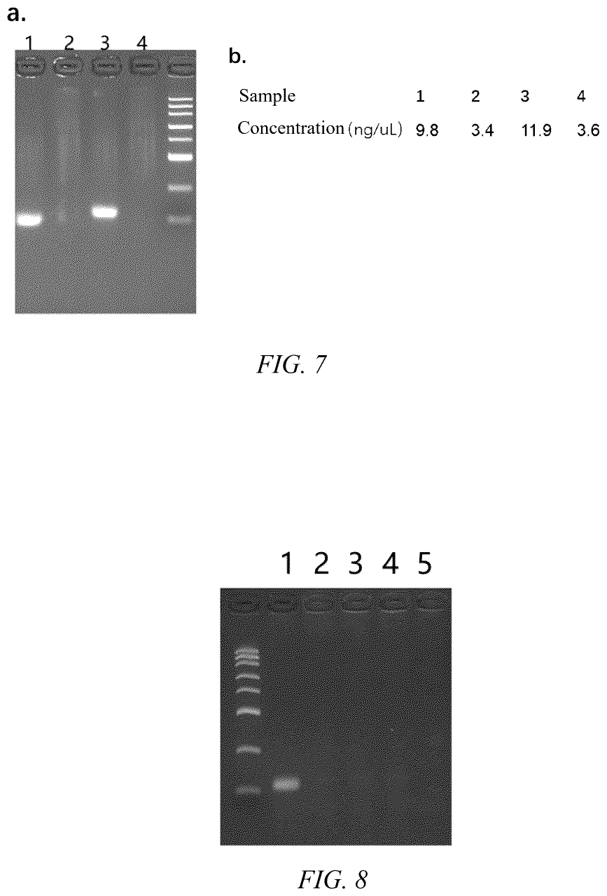

For the gel electrophoresis test, 2 μL of purified PCR product samples were mixed well with 0.5 μL of a dye for plasmid mass extraction (Goldview, SBS GENETECH CO., LTD.), the resulting mixed solution was added into the well of 2.5% agarose gel (Invitrogen) for gel electrophoresis, and the gel image (DYY-8C type, BEIJING LIUYI BIOTECHNOLOGY CO., LTD; 120V, 20 min) was collected. As shown in FIG. 7a, lanes 1 and 3 show purified PCR products with the target sequence as a template, and lanes 2 and 4 show purified PCR products with the single-base mutation sequence as a template. It can be seen that lanes 1 and 3 show obvious target bands, while the mutation sequence does not produce a band. As shown in FIG. 7b, the concentrations of these purified products can be measured with Thermo Scientific™ NanoDrop™ One Microvolume UV-Vis Spectrophotometers.

### Example 5: Detection of Unknown Mutants of Single-Base Mutations Using Unbalanced PCR

The target sequence to be detected was SEQ ID NO: 1 in example 1; the target sequence was replaced by distilled water as a negative control; and the short-chain primer 1 used was 5′-ATCGAGATTTCT (SEQ ID NO: 3), 5′-ATCGAGATTTCA (SEQ ID NO: 4), 5′-ATCGAGATTTCG (SEQ ID NO: 5) or 5′-ATCGAGATTTCC (SEQ ID NO: 6), and the long-chain primer 2 used was 5′-CTTTACTTACTACACCTCAG (SEQ ID NO: 19).

PCR amplification conditions were as follows: the reaction system was 20 μL, including 7 μL of ddH₂O, 1 μL of primer 1 (10 μmol·L⁻¹) (one PCR was performed for each primer 1), 1 μL of primer 2 (10 μmol·L⁻¹), 1 μL of the target sequence as a template DNA (1 nmol·L⁻¹), and 10 μL of HiFi-KAPA polymerase 2×.

PCRs were performed on a Biometra T1 thermolcycler. The reaction conditions involved: pre-denaturation at 98° C. for 30 s; denaturation at 98° C. for 10 s, annealing at a particular temperature (45° C. or 50° C.) for 30 s, and extension at 72° C. for 15 s, 20 cycles; and extension at 72° C. for 5 min.

Gel electrophoresis test: 2 μL of PCR product samples were mixed well with 0.5 μL of a dye for plasmid mass extraction (Goldview, SBS GENETECH CO., LTD.), the resulting mixed solution was added into the well of 2.5% agarose gel (Invitrogen) for gel electrophoresis, and the gel image (DYY-8C type, BEIJING LIUYI BIOTECHNOLOGY CO., LTD; 120V, 20 min) was collected.

The results are shown in FIG. 8. Lane 1 shows the result using SEQ ID NO: 3 as primer 1, lane 2 shows the result using SEQ ID NO: 4 as primer 1, lane 3 shows the result using SEQ ID NO: 5 as primer 1, lane 4 shows the result using SEQ ID NO: 6 as primer 1, lane 5 shows a blank control in which the target sequence is replaced with water, and the leftmost lane shows the molecular weight marker. It can be seen that the target band appears only when SEQ ID NO: 3, which completely matches the template DNA, is used as the primer. That is, by using 4 primers with 4 different bases at a position of pairing with a target mutation site, the unbalanced PCR method of the present application can be used to accurately determine the bases at the target site, indicating that the method of the present application can identify single-base mutants very accurately.

### Example 6: Detection of Single-Base Mutations by Real-Time Fluorescent Quantitative PCR

In this example, the unbalanced PCR method was applied to the real-time fluorescent quantitative PCR. The target sequence to be detected was SEQ ID NO: 1 in example 1, the sequence having a single-base mutation relative to the target sequence was SEQ ID NO: 2 in example 1, the short-chain primer 1 used was 5′-ATCGAGATTTCT (SEQ ID NO: 3), and the long-chain primer 2 used was 5′-CTTTACTTACTACACCTCAG (SEQ ID NO: 19). A blank control was set up by replacing the template sequence with water.

qPCR amplification conditions were as follows: the reaction system was 20 μL, including 6 μL of ddH₂O, 1 μL of primer 1 (10 μmol·L⁻¹), 1 μL of primer 2 (10 μmol·L⁻¹), 1 μL of template DNA (the target sequence or the sequence having a single-base mutation relative to the target sequence) (1 nmol·L⁻¹), 10 μL of DNA polymerase (HiFi-KAPA polymerase 2×), and 1 μL of SYBR Green I (20×) (KGM030, KEYGEN BIOTECH CO., LTD.).

The real-time fluorescent quantitative PCR was performed on a qPCR instrument (model: QuantStudio 5, manufacturer: ABI). The reaction conditions were as follows: pre-denaturation at 98° C. for 30 s; denaturation at 98° C. for 10 s, annealing at a particular temperature (45° C. or 50° C.) for 30 s, and extension at 72° C. for 15 s, 20 cycles; and extension at 72° C. for 5 min. In order to obtain the solubility curve, the reaction was further performed at 95° C. for 15 s and at 60° C. for 1 minute, and the denaturation was performed at 95° C. for 1 second.

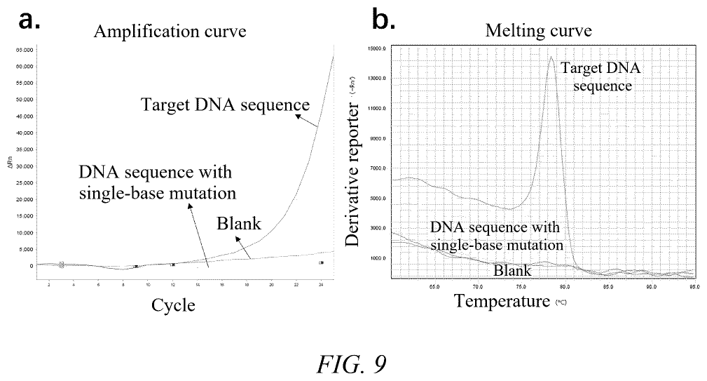

As can be seen from FIG. 9a, the fluorescence signal of the reaction using the target sequence as a template increased significantly, while the signals of the single-base mutation sequence and the blank control were very weak or did not increase significantly. FIG. 9b shows a melting curve, wherein the peak position of the target sequence indicates that a correct product was obtained using the method of the present application. This example illustrates that the method of the present application can be applied to the fluorescent quantitative PCR for the detection of single-base mutations.

The embodiments of the present invention are not limited to the above-mentioned examples. Without departing from the spirit and scope of the present invention, those skilled in the art can make various changes and improvements to the present invention in forms and details, and all these changes and improvements are contemplated to be within the scope of protection of the present invention.

1. A method for detecting whether there is a mutation at an expected single-base mutation site of a nucleic acid sequence, the method comprising:
providing a sample containing a nucleic acid sequence to be detected;
amplifying the nucleic acid sequence to be detected in the sample by a polymerase chain reaction using a primer group, wherein the primer group comprises the following primers:
an identification primer comprising from the 5′ end to the 3′ end: (a) a nucleotide sequence which complements a segment of continuous nucleotides in the nucleic acid sequence to be detected, wherein the 5′ end of the continuous nucleotides starts at a first nucleotide downstream of an expected mutation site, and (b) a nucleotide which complements a non-mutated nucleotide at the expected single-base mutation site of the nucleic acid sequence to be detected,
an amplification primer capable of amplifying an amplification product obtained by amplifying the nucleic acid sequence to be detected using the identification primer,
wherein the identification primer is 1 to 19 nucleotides less than the amplification primer; and
detecting whether there is a specific amplification product in a reaction product, wherein the presence of the specific amplification product indicates that there is no single-base mutation at the expected mutation site of the nucleic acid sequence to be detected.

2. A method for detecting a nucleotide at an expected single-base mutation site of a nucleic acid sequence, the method comprising:
providing a sample containing a nucleic acid sequence to be detected;
amplifying the nucleic acid sequence in the sample by a polymerase chain reaction using a primer group, wherein the primer group comprises the following primers:
an identification primer, which is composed of, from the 5′ end to the 3′ end, (a) a nucleotide sequence which complements a segment of continuous nucleotides in the nucleic acid sequence to be detected, wherein the 5′ end of the continuous nucleotides starts at a first nucleotide downstream of an expected mutation site, and (b) a nucleotide which complements a nucleotide expected to exist at the expected single-base mutation site of the nucleic acid sequence to be detected, and
an amplification primer capable of amplifying an amplification product obtained by amplifying the nucleic acid sequence to be detected using the identification primer,
wherein the identification primer is 1 to 19 nucleotides less than the amplification primer; and
detecting whether there is a specific amplification product in a reaction product, wherein the presence of the specific amplification product indicates that there is the nucleotide expected to exist at the expected mutation site of the nucleic acid sequence to be detected.

3. The method according to claim 1, wherein the identification primer is 2 to 16 nucleotides less than the amplification primer.

4. The method according to claim 1, wherein the identification primer is 11 to 16 nucleotides in length.

5. The method according to claim 1, wherein the amplification primer is 15 to 25 nucleotides in length.

6. The method according to claim 1, wherein the identification primer is 12 nucleotides in length and the amplification primer is 20 nucleotides in length, or the identification primer is 15 nucleotides in length and the amplification primer is 20 nucleotides in length.

7. (canceled)

8. The method according to claim 1, wherein the polymerase chain reaction is performed using a DNA polymerase selected from: a hot-start Taq polymerase, a TaqNova Stoffel DNA polymerase, an HiFi-KAPA polymerase and an Hemo KlenTaq polymerase.

9. The method according to claim 1, wherein the polymerase chain reaction is performed at an annealing temperature of 44° C. to 52° C.

10. The method according to claim 1, wherein the polymerase chain reaction is performed at an annealing temperature of 45° C. to 50° C.

11. (canceled)

12. The method according to claim 1, wherein the polymerase chain reaction is a fluorescent quantitative PCR.

13. (canceled)

14. A primer group for detecting a single-base mutation in a nucleic acid sequence, the primer group comprising the following primers:
an identification primer, which is composed of, from the 5′ end to the 3′ end, (a) a nucleotide sequence which complements a segment of continuous nucleotides in a nucleic acid sequence to be detected, wherein the 5′ end of the continuous nucleotides starts at a first nucleotide downstream of an expected mutation site; and (b) a nucleotide which complements a non-mutated nucleotide or an expected mutated nucleotide at an expected single-base mutation site of a nucleic acid sequence to be detected;
an amplification primer capable of amplifying an amplification product obtained by amplifying the nucleic acid sequence to be detected using the identification primer,
wherein the identification primer is 1 to 19 nucleotides less than the amplification primer.

15. The primer group according to claim 14, wherein the identification primer is 11 to 16 nucleotides in length.

16. The primer group according to claim 14, wherein the amplification primer is 15 to 25 nucleotides in length.

17. The primer group according to claim 14, wherein the identification primer is 12 nucleotides in length and the amplification primer is 20 nucleotides in length, or the identification primer is 15 nucleotides in length and the amplification primer is 20 nucleotides in length.

18. Use of the primer group according to claim 14 in the preparation of a mixture, a kit or a biological detection device for detecting a single-base mutation in a nucleic acid sequence.

19. (canceled)

20. A kit for detecting a single-base mutation in a nucleic acid sequence, comprising the primer group according to claim 14.

21. (canceled)

22. A biological detection device for detecting a single-base mutation in a nucleic acid sequence, comprising the primer group according to claim 14.

23. (canceled)

24. The kit according to claim 20, wherein the DNA polymerase is selected from: a hot-start Taq polymerase, a TaqNova Stoffel DNA polymerase, an HiFi-KAPA polymerase and an Hemo KlenTaq polymerase.

25. The kit according to claim 20, further comprising a reagent for detecting an amplification product.

26. The method according to claim 2, wherein the identification primer is 2 to 16 nucleotides less than the amplification primer.


SEQUENCE LISTING (26 sequences)

SEQ ID NO: 1 - DNA, 98 bp, Artificial Sequence
  Target sequence
ctttacttactacacctcagatatatttcttcatgaagacctcacagtaaaaataggtgattttggtctagctacagaagaaatctcgatggagtggg

SEQ ID NO: 2 - DNA, 98 bp, Artificial Sequence
  SNP sequence
ctttacttactacacctcagatatatttcttcatgaagacctcacagtaaaaataggtgattttggtctagctacagatgaaatctcgatggagtggg

SEQ ID NO: 3 - DNA, 12 bp, Artificial Sequence
  1-12nt-1
atcgagatttct

SEQ ID NO: 4 - DNA, 12 bp, Artificial sequence
  1-12nt-2
atcgagatttca

SEQ ID NO: 5 - DNA, 12 bp, Artificial sequence
  1-12nt-3
atcgagatttcg

SEQ ID NO: 6 - DNA, 12 bp, Artificial sequence
  1-12nt-4
atcgagatttcc

SEQ ID NO: 7 - DNA, 13 bp, Artificial sequence
  1-13nt-1
catcgagatttct

SEQ ID NO: 8 - DNA, 13 bp, Artificial sequence
  1-13nt-2
catcgagatttca

SEQ ID NO: 9 - DNA, 13 bp, Artificial sequence
  1-13nt-3
catcgagatttcg

SEQ ID NO: 10 - DNA, 13 bp, Artificial sequence
  1-13nt-4
catcgagatttcc

SEQ ID NO: 11 - DNA, 14 bp, Artificial sequence
  1-14nt-1
ccatcgagatttct

SEQ ID NO: 12 - DNA, 14 bp, Artificial sequence
  1-14nt-2
ccatcgagatttca

SEQ ID NO: 13 - DNA, 14 bp, Artificial sequence
  1-14nt-3
ccatcgagatttcg

SEQ ID NO: 14 - DNA, 14 bp, Artificial sequence
  1-14nt-4
ccatcgagatttcc

SEQ ID NO: 15 - DNA, 15 bp, Artificial sequence
  1-15nt-1
tccatcgagatttct

SEQ ID NO: 16 - DNA, 15 bp, Artificial sequence
  1-15nt-2
tccatcgagatttca

SEQ ID NO: 17 - DNA, 15 bp, Artificial sequence
  1-15nt-3
tccatcgagatttcg

SEQ ID NO: 18 - DNA, 15 bp, Artificial sequence
  1-15nt-4
tccatcgagatttcc

SEQ ID NO: 19 - DNA, 20 bp, Artificial sequence
  2-20nt
ctttacttactacacctcag

SEQ ID NO: 20 - DNA, 20 bp, Artificial sequence
  Ordinary primer 1
cccactccatcgagatttct

SEQ ID NO: 21 - DNA, 20 bp, Artificial sequence
  Ordinary primer 2
ctttacttactacacctcag

SEQ ID NO: 22 - DNA, 12 bp, Artificial sequence
  2-12nt
ctttacttacta

SEQ ID NO: 23 - DNA, 11 bp, Artificial sequence
  1-11nt-1
tcgagatttct

SEQ ID NO: 24 - DNA, 11 bp, Artificial sequence
  1-11nt-2
tcgagatttca

SEQ ID NO: 25 - DNA, 11 bp, Artificial sequence
  1-11nt-3
tcgagatttcg

SEQ ID NO: 26 - DNA, 11 bp, Artificial sequence
  1-11nt-4
tcgagatttcc